(19) United States
(12) Patent Application Publication
(10) Pub. No.: US 2022/0240755 A1
(43) Pub. Date: Aug. 4, 2022

(54) PUSH BUTTON MECHANISM FOR ENDOSCOPE, AND ENDOSCOPE

(71) Applicants: OLYMPUS CORPORATION, Tokyo (JP); ALPS ALPINE CO., LTD., Tokyo (JP)

(72) Inventors: Shunsuke KIMURA, Aizuwakamatsu-shi (JP); Reiji KOYAMA, Kawagoe-shi (JP); Tetsu NUMATA, Miyagi (JP); Shigeto GORAI, Miyagi (JP); Akihiro KUSAKA, Miyagi (JP)

(73) Assignees: OLYMPUS CORPORATION, Tokyo (JP); ALPS ALPINE CO., LTD., Tokyo (JP)

(21) Appl. No.: 17/725,891

(22) Filed: Apr. 21, 2022

Related U.S. Application Data
(63) Continuation of application No. PCT/JP2020/039680, filed on Oct. 22, 2020.

(30) Foreign Application Priority Data
Oct. 29, 2019 (JP) 2019-196704

(51) Int. Cl. A61B 1/005; H01H 13/14; A61B 1/00

(52) U.S. Cl. CPC A61B 1/0052; A61M 25/0136; A61B 1/00042; H01H 13/14

(57) ABSTRACT

A push button mechanism for an endoscope includes: a first stem having a first longitudinal direction and having a first end portion connected inside a finger placement member; a second stem having a first end face in contact with a second end portion of the first stem and slidably provided along a second longitudinal direction having a predetermined angle relative to the first longitudinal direction; a switch configured to be switched by the second stem sliding in the second longitudinal direction in contact with the second end portion of the first stem moving in conjunction with the finger placement member; and an immobile member including a space in which the second end portion of the first stem is housed, and the space has a sectional shape similar to the second end portion formed in a deformed shape and prevents rotation of the first stem.

### CROSS REFERENCE TO RELATED APPLICATION

This application is a continuation application of PCT/JP2020/039680 filed on Oct. 22, 2020 and claims benefit of Japanese Application No. 2019-196704 filed in Japan on Oct. 29, 2019, the entire contents of which are incorporated herein by this reference.

### BACKGROUND OF THE INVENTION

### 1. Field of the Invention

The present invention relates to a push button mechanism for an endoscope and the endoscope, the push button mechanism being provided at, for example, an operation portion.

### 2. Description of the Related Art

As is well known, an endoscope is widely used for observation, treatment, and the like inside a living body (inside a body cavity), and for examination, repair, and the like inside an industrial plant facility. Such an endoscope includes an operation portion that is grasped by a user, and the operation portion is provided with, for example, a bending operation knob for bending a bending portion provided at an insertion portion, and a push button for operating an external instrument such as a light source device or a video processor. For example, a push button device for an endoscope as disclosed in Japanese Patent Application Laid-Open Publication No. 2014-117574 is well known as a push button provided to such an endoscope.

### SUMMARY OF THE INVENTION

A push button mechanism for an endoscope according to an aspect of the present invention includes: a finger placement member having elasticity and disposed at an operation portion of the endoscope; a first stem having a first longitudinal direction and having a first end portion connected inside the finger placement member; a second stem having a first end face in contact with a second end portion of the first stem and slidably provided along a second longitudinal direction having a predetermined angle relative to the first longitudinal direction, the second end portion being opposite to the first end portion; a switch configured to be switched by the second stem sliding in the second longitudinal direction in contact with the second end portion of the first stem moving in conjunction with pushing of the finger placement member; and an immobile member including a space in which the second end portion of the first stem is housed, and the space has a sectional shape similar to the second end portion formed in a deformed shape and prevents rotation of the first stem.

A push button mechanism for an endoscope according to another aspect of the present invention includes: a finger placement member having elasticity and disposed at an operation portion of the endoscope; a first stem having a first longitudinal direction and having a first end portion connected inside the finger placement member; a second stem having a first end face in contact with a second end portion of the first stem and slidably provided along a second longitudinal direction having a predetermined angle relative to the first longitudinal direction, the second end portion being opposite to the first end portion; and a switch configured to be switched by the second stem sliding in the second longitudinal direction in contact with the second end portion of the first stem moving in conjunction with pushing of the finger placement member, a contact surface of the second end portion in contact with the first end face has a convex spherical surface shape, and the first end face is a flat surface orthogonal to the second longitudinal direction.

An endoscope according to an aspect of the present invention includes: a finger placement member having elasticity and disposed at an operation portion that is grasped by a user; a first stem having a first longitudinal direction and having a first end portion connected inside the finger placement member; a second stem having a first end face in contact with a second end portion of the first stein and slidably provided along a second longitudinal direction having a predetermined angle relative to the first longitudinal direction, the second end portion being opposite to the first end portion; a switch configured to be switched by the second stem sliding in the second longitudinal direction in contact with the second end portion of the first stem moving in conjunction with pushing of the finger placement member; and an immobile member including a space in which the second end portion of the first stem is housed, and the space has a sectional shape similar to the second end portion formed in a deformed shape and prevents rotation of the first stem.

An endoscope according to another aspect of the present invention includes: a finger placement member having elasticity and disposed at an operation portion that is grasped by a user; a first stem having a first longitudinal direction and having a first end portion connected inside the finger placement member; a second stem having a first end face in contact with a second end portion of the first stem and slidably provided along a second longitudinal direction having a predetermined angle relative to the first longitudinal direction, the second end portion being opposite to the first end portion; and a switch configured to be switched by the second stem sliding in the second longitudinal direction in contact with the second end portion of the first stem moving in conjunction with pushing of the finger placement member, a contact surface of the second end portion in contact with the first end face has a convex spherical surface shape, and the first end face is a flat surface orthogonal to the second longitudinal direction.

### BRIEF DESCRIPTION OF THE DRAWINGS

FIG. 1 is a diagram illustrating appearance of an endoscope in which a push button mechanism according to an aspect of the present invention is provided at an operation portion;

FIG. 2 is a cross-sectional view illustrating a configuration of the push button mechanism;

FIG. 3 is a cross-sectional view of a switch unit along line III-III in FIG. 2;

FIG. 4 is a cross-sectional view of the switch unit along line IV-IV in FIG. 2;

FIG. 5 is a cross-sectional view illustrating a state in which two push button mechanisms are disposed adjacent to each other; and

FIG. 6 is a cross-sectional view illustrating a state in which a push button mechanism is disposed adjacent to another internal component.

### DETAILED DESCRIPTION OF THE PREFERRED EMBODIMENTS

The following description will be made with an example of an endoscope including a push button mechanism of the present invention. Note that, in the following description, drawings based on each embodiment are schematic, and relations between a thickness and a width of each part, thickness ratios of parts, and the like may be different from actual values, Dimensional relations between elements and ratios of elements may also differ between the drawings in some cases.

An endoscope in the following configuration explanation will be described with an example of what is called a flexible endoscope in which an insertion portion has flexibility for insertion into a digestive system in an upper portion or a lower portion of a living body, but is not limited to a flexible endoscope and is a technology that is also applicable to what is called a rigid endoscope that includes a rigid insertion portion and is used for surgery, and an industrial endoscope for examining an engine, a plant, or the like.

A push button mechanism for an endoscope according to an aspect of the present invention will be described below with reference to the accompanying drawings.

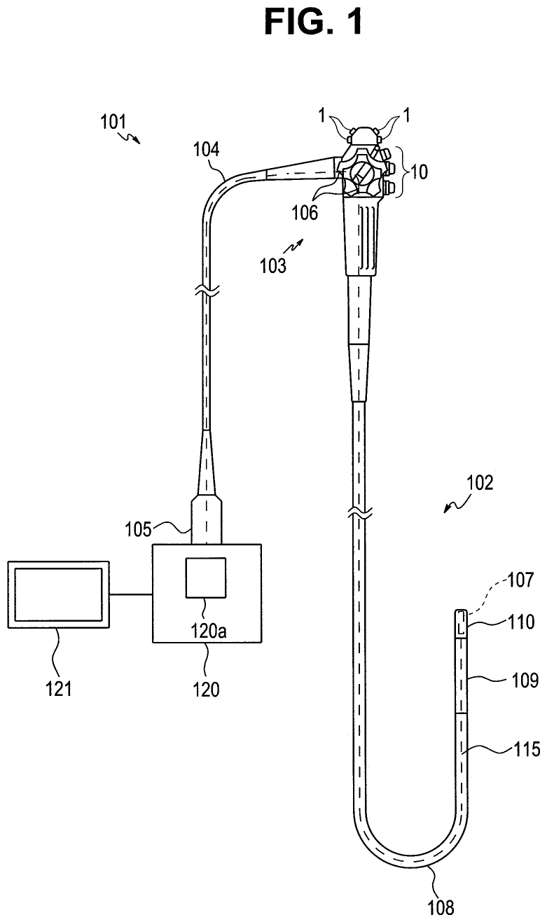

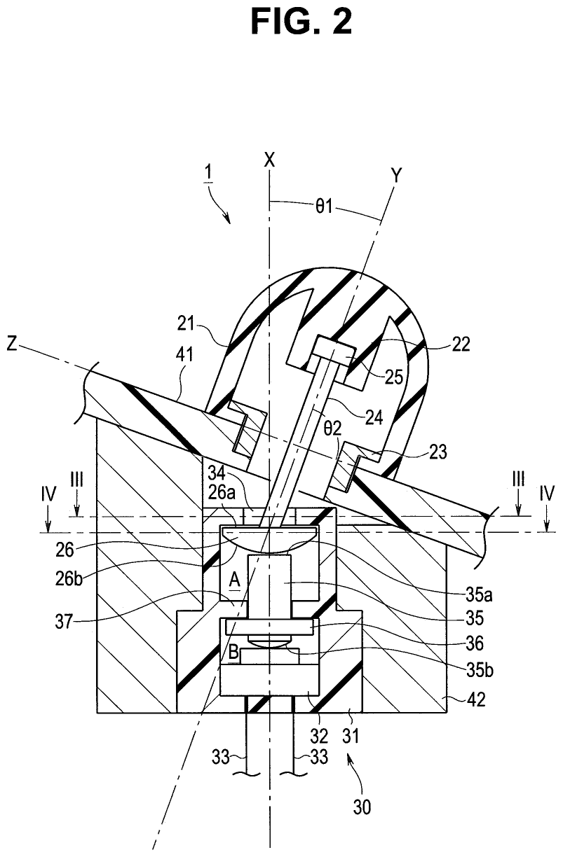

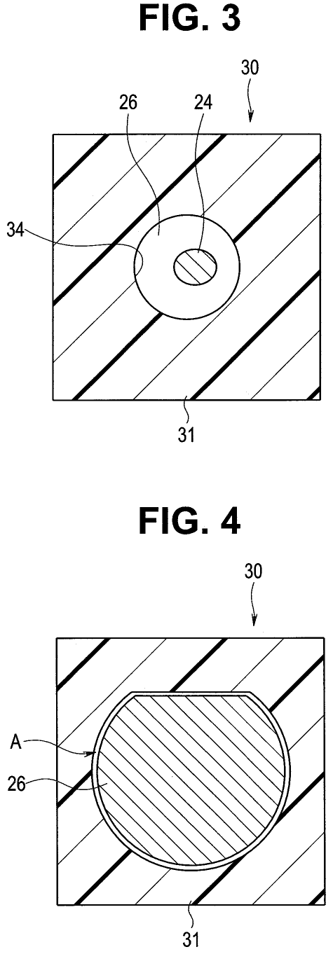

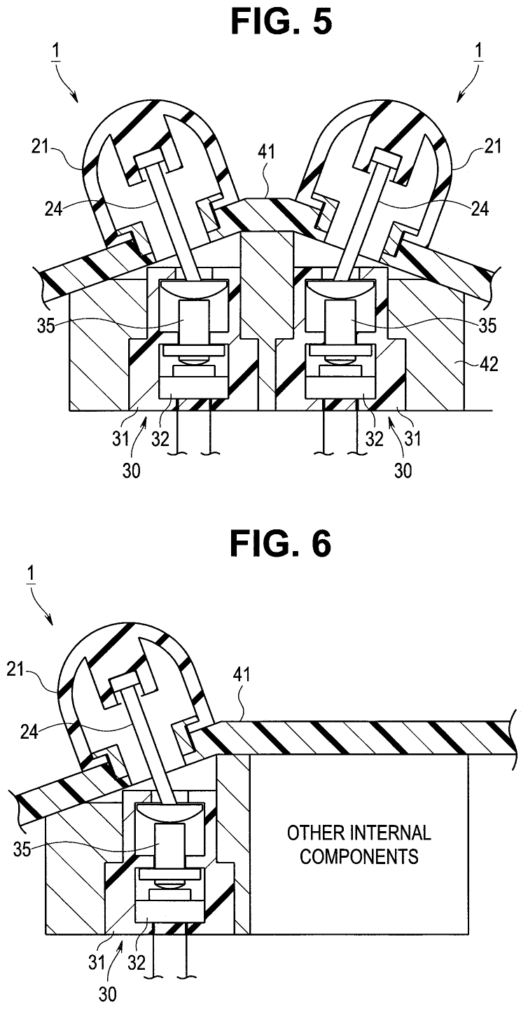

FIG. 1 is a diagram illustrating appearance of an endoscope in which the push button mechanism according to the aspect of the present invention is provided at an operation portion, FIG. 2 is a cross-sectional view illustrating a configuration of the push button mechanism, FIG. 3 is a cross-sectional view of a switch unit along line III-III in FIG. 2, FIG. 4 is a cross-sectional view of the switch unit along line IV-IV in FIG. 2, FIG. 5 is a cross-sectional view illustrating a state in which two push button mechanisms are disposed adjacent to each other, and FIG. 6 is a cross-sectional view illustrating a state in which a push button mechanism is disposed adjacent to another internal component.

First, an example of a configuration of an endoscope 101 in which a push button unit 1 as a push button mechanism for an endoscope according to the present invention is provided at an operation portion 103 will be described below with reference to FIG. 1.

The endoscope 101 of the present embodiment can be introduced into a subject such as a human body and is configured to optically pick up an image of a predetermined observation site in the subject.

Note that the subject into which the endoscope 101 is introduced is not limited to a human body but may be any other living body or may be an artificial object such as a machine or a building.

The endoscope 101 mainly includes an insertion portion 102 that is introduced into the subject, the operation portion 103 positioned at a proximal end of the insertion portion 102, and a universal code 104 extending from a side part of the operation portion 103.

The insertion portion 102 has a configuration in which a distal end portion 110, a bending portion 109, and a flexible tube portion 108 are continuously provided, the distal end portion 110 being disposed at a distal end, the bending portion 109 being bendable and disposed on a proximal end side of the distal end portion 110, the flexible tube portion 108 having flexibility, being disposed on the proximal end side of the bending portion 109, and connected to the distal end side of the operation portion 103.

An image pickup unit 107 is provided at the distal end portion 110. An angle operation knob 106 for operating bending of the bending portion 109 is provided at the operation portion 103.

An endoscope connector 105 that is connected to an external device 120 is provided at a proximal end portion of the universal code 104. The external device 120 connected to the endoscope connector 105 is connected to an image display unit 121 such as a monitor through a cable.

The endoscope 101 also includes a composite cable 115 (not illustrated) inserted in the universal code 104, the operation portion 103, and the insertion portion 102, and includes an optical fiber bundle (not illustrated in the drawing) that transmits illumination light from a light source unit provided at the external device 120.

The composite cable 115 electrically connects the endoscope connector 105 and the image pickup unit 107. When the endoscope connector 105 is connected to the external device 120, the image pickup unit 107 is electrically connected to the external device 120 through the composite cable 115.

Electric power supply from the external device 120 to the image pickup unit 107 and communication between the external device 120 and the image pickup unit 107 are performed through the composite cable 115.

An image processing unit is provided at the external device 120. The image processing unit generates a video signal based on an image-pickup-device output signal outputted from the image pickup unit 107, and outputs the video signal to the image display unit 121. Specifically, in the present embodiment, an optical image (endoscope image) picked up by the image pickup unit 107 is displayed as a video on the image display unit 121.

Note that the endoscope 101 does not necessarily need to be connected to the external device 120 or the image display unit 121 but may include, for example, part or entire of the image processing unit or the monitor.

A light guide (not illustrated in the drawing) to be described later as the optical fiber bundle transmits light emitted from the light source unit of the external device 120 to an illumination window as an illumination light emission portion of the distal end portion 110. The light source unit may be disposed at the operation portion 103 or the distal end portion 110 of the endoscope 101.

At the operation portion 103, operation buttons 10 such as an air-water feeding button and a suction button, and the push button units 1 as a plurality of push button devices for the endoscope for performing releasing and various operation instructions are provided in addition to the angle operation knob 106.

The push button units 1 are an endoscope operation means for performing, for the external device 120, remote operations of a VTR, a video printer, a video disk, and the like and instructions of image freezing, releasing, metering mode control, size change of an output screen on the monitor.

Subsequently, each push button unit 1 as a push button mechanism for the endoscope according to the present embodiment will be described below in detail.

As illustrated in FIG. 2, the push button unit 1 mainly includes a rubber cap 21 as a finger placement member having elasticity, a first stem 24 as a first operation rod, a second stem 35 as a second operation rod, and a switch unit 30.

A stem connection portion 22 in a tubular shape is integrally formed inside the rubber cap 21, and an outer peripheral end portion of the rubber cap 21 in an inward flange shape is sandwiched between an exterior case 41 of the operation portion 103 and a fastening pipe 23, for example, screwed to the exterior case 41 so that the rubber cap 21 is held watertight and disposed on the exterior case 41.

The first stem 24 includes an end portion 25 in an outward flange shape at one end as an example of a first end portion and includes a disk portion 26 at the other end as an example of a second end portion opposite to the one end. The first stem 24 is connected to the rubber cap 21 with the end portion 25 as an upper end portion of the first stem 24 being housed in the stem connection portion 22 formed in a bottomed tubular shape inside the rubber cap 21.

Note that the disk portion 26 provided at a lower end portion of the first stem 24 is housed in an internal space A at an upper portion of the switch unit 30.

In the switch unit 30, a switch portion 32 including an elastic contact point such as a tactile switch is provided in an internal space B at a lower portion of a case 31 as an immobile member, and the second stem 35 is slidably housed above the switch portion 32 in the case 31. The switch unit 30 is fixed to an interior decorative frame 42 as an immobile member fixed to the exterior case 41 of the operation portion 103.

In the case 31, an opening portion 34 through which the first stem 24 is inserted is formed at an upper portion. The disk portion 26 provided to the first stem 24 is housed in the internal space A at the upper portion of the case 31 and contacts an inner wall of the upper portion of the case 31, through which the opening portion 34 is formed, thereby preventing removal of the first stem 24 from the case 31.

An inward flange 37 through which the second stem 35 is inserted and that an outward flange 36 of the second stem 35 contacts is provided at a middle part in the case 31. Note that the outward flange 36 of the second stem 35 is disposed in the internal space B at the lower portion of the case 31 in which the switch portion 32 is disposed.

The switch portion 32 is electrically connected to a lead line 33 extending from the lower portion of the case 31. The lead line 33 is electrically connected to an electric contact portion provided to the endoscope connector 105.

In the push button unit 1 described above, an axis X and an axis Y are set to have a predetermined angle θ1, the axis X being in a direction of a switch operation to turn on or off the switch portion 32 and a direction (example of a second longitudinal direction) in which the second stem 35 slides along a longitudinal direction of the second stem 35, the axis Y being in a longitudinal direction (example of a first longitudinal direction) of the first stem 24.

In other words, in the push button unit 1, the axis Y of the first stem 24 has the predetermined angle θ1 relative to the axis X of the second stem 35 that moves in a pushing direction of a switch operation to turn on or off the switch portion 32.

Moreover, in the push button unit 1, the first stem 24 is disposed with the axis Y at a predetermined angle θ2 relative to a flat surface (an axis Z in the drawing) as a surface of the exterior case 41 of the operation portion 103. Note that, in this example, the first stem 24 is provided so that the axis Y is substantially orthogonal) 90° to the flat surface (the axis Z in the drawing) of the exterior case 41.

As illustrated in FIGS. 2 and 3, the axis Y of the first stem 24 extends not in a perpendicular (90°) direction relative to an upper surface 26a of the disk portion 26 provided at the lower end portion but at an angle (90°−θ1), and the disk portion 26 is provided at a tilt.

As illustrated in FIG. 4, the disk portion 26 has a sectional shape of what is called a D-cut shape that is a deformed shape in which a part of an outer periphery is cut out straight. Accordingly, a hole section of the internal space A of the case 31 of the switch unit 30 is a rotation preventing shape that is a sectional shape of a D-cut shape similar to the D-cut shape of the disk portion 26. When prevented from rotating, the first stem 24 is smoothly pushed in accordance with motion of pushing the rubber cap 21, and thus a switch operation defect is prevented.

Note that, in the disk portion 26 of the first stem 24, the upper surface 26a has a flat surface shape and a lower surface 26b as a contact surface has a convex spherical surface shape. In the second stem 35, an upper surface 35a as an example of a first end face that the lower surface 26b of the disk portion 26 contacts has a flat surface shape orthogonal to the longitudinal axis X, and a lower surface 35b that pushes the switch portion 32 has a convex spherical surface shape.

The convex spherical lower surface 26h of the disk portion 26 of the first stem 24 is larger than an outline of the upper surface 35a of the second stem 35, in other words, the convex spherical lower surface 26h has a surface area larger than a surface area of the upper surface 35a of the second stem 35.

In the push button unit 1, the first stein 24 moves in conjunction with pushing of the rubber cap 21 by a finger of a user and is pushed so that the upper surface 35a of the second stem 35 contacts the lower surface 26h of the disk portion 26. Then, the second stem 35 is pushed in the longitudinal direction along the axis X and moved downward so that the switch portion 32 is turned on or off.

Lengths of the first stem 24 and the second stem 35 are set so that a position at which the lower surface 26b of the disk portion 26 of the first stem 24 contacts the upper surface 35a of the second stem 35 is inside the operation portion 103 with respect to the surface of the exterior case 41 of the operation portion 103.

Note that, since the lower surface 26b of the disk portion 26 of the first stem 24, which contacts the upper surface 35a of the second stem 35, has a convex spherical surface, the push button unit 1 has such a configuration that the switch portion 32 can be turned on or off in any of a direction from top along the axis Y as the longitudinal direction of the first stem 24 in an initial state, a side direction orthogonal to the axis Y, and an oblique direction at an angle relative to the axis Y.

In the push button unit 1 as a push button mechanism for an endoscope according to the present embodiment described above, the first stem 24 and the second stem 35 are disposed with the predetermined angle θ1 between the axis Y and the axis X, and thus the longitudinal direction of the second stem 35 and an orientation of the switch unit 30 as an immobile member do not need to be aligned for the rubber cap 21 and the first stem 24.

In other words, in the push button unit 1, orientations of the second stem 35 and the switch unit 30 as an immobile member can be changed relative to the rubber cap 21 and the first stem 24.

Accordingly, the endoscope 101 has an increased degree of freedom of a position at which the push button unit 1 is disposed at the operation portion 103, and also has an increased degree of freedom of arrangement of a space in which the switch unit 30 of the push button unit 1 is housed in the operation portion 103. As a result, increase of a size of the operation portion 103 of the endoscope 101 can be prevented, and the size can be reduced depending on arrangement with any other internal component in the operation portion 103.

For example, a plurality, two in this example, of push button units 1 can be disposed close to each other as illustrated in FIG. 5, and each push button unit 1 can be installed near another internal component without interference with the other internal component as illustrated in FIG. 6, and thus the size of the operation portion 103 of the endoscope 101 is prevented from increasing and can be reduced.

The invention described above in the embodiment is not limited to the embodiment and the modifications but may include various kinds of other modifications without departing from the gist of the invention when performed. Moreover, inventions at various kinds of stages are included in the above-described embodiment, and various kinds of inventions can be extracted as appropriate combinations among a plurality of disclosed components.

For example, some components may be deleted from among all components described in the embodiment, and a configuration from which the components are deleted may be extracted as an invention as long as a described problem can be solved and described effects can be obtained.

According to the present invention, it is possible to provide a small-sized push button mechanism for an endoscope in which size increase of an operation portion is prevented.

What is claimed is:

1. A push button mechanism for an endoscope, the push button mechanism comprising:
a finger placement member having elasticity and disposed at an operation portion of the endoscope;
a first stem having a first longitudinal direction and having a first end portion connected inside the finger placement member;
a second stem having a first end face in contact with a second end portion of the first stem and slidably provided along a second longitudinal direction having a predetermined angle relative to the first longitudinal direction, the second end portion being opposite to the first end portion;
a switch configured to be switched by the second stem sliding in the second longitudinal direction in contact with the second end portion of the first stem moving in conjunction with pushing of the finger placement member; and
an immobile member including a space in which the second end portion of the first stem is housed,
wherein the space has a sectional shape similar to the second end portion formed in a deformed shape and prevents rotation of the first stem.

2. The push button mechanism for an endoscope according to claim wherein the first stem is disposed so that the first longitudinal direction is substantially orthogonal to a surface of an exterior of the operation portion.

3. The push button mechanism for an endoscope according to claim 1, wherein lengths of the first stem and the second stem are set so that the second end portion and the first end face contact each other at a position inside a surface of an exterior of the endoscope.

4. The push button mechanism for an endoscope according to claim 1, wherein lengths of the first stem and the second stem are set so that the second end portion and the first end face contact each other in the space of the immobile member.

5. An endoscope comprising:
a finger placement member having elasticity and disposed at an operation portion that is grasped by a user;
a first stem having a first longitudinal direction and having a first end portion connected inside the finger placement member;
a second stem having a first end face in contact with a second end portion of the first stem and slidably provided along a second longitudinal direction having a predetermined angle relative to the first longitudinal direction, the second end portion being opposite to the first end portion;
a switch configured to be switched by the second stein sliding in the second longitudinal direction in contact with the second end portion of the first stem moving in conjunction with pushing of the finger placement member; and
an immobile member including a space in which the second end portion of the first stem is housed,
wherein the space has a sectional shape similar to the second end portion formed in a deformed shape and prevents rotation of the first stem.

6. A push button mechanism for an endoscope, the push button mechanism comprising:
a finger placement member having elasticity and disposed at an operation portion of the endoscope;
a first stem having a first longitudinal direction and having a first end portion connected inside the finger placement member;
a second stem having a first end face in contact with a second end portion of the first stem and slidably provided along a second longitudinal direction having a predetermined angle relative to the first longitudinal direction, the second end portion being opposite to the first end portion; and
a switch configured to be switched by the second stem sliding in the second longitudinal direction in contact with the second end portion of the first stem moving in conjunction with pushing of the finger placement member, wherein
a contact surface of the second end portion in contact with the first end face has a convex spherical surface shape, and
the first end face is a flat surface orthogonal to the second longitudinal direction.

7. The push button mechanism for an endoscope according to claim 6, wherein the first stem is disposed so that the first longitudinal direction is substantially orthogonal to a surface of an exterior of the operation portion.

8. The push button mechanism for an endoscope according to claim 6, wherein lengths of the first stem and the second stem are set so that the second end portion and the first end face contact each other at a position inside a surface of an exterior of the endoscope.

9. The push button mechanism for an endoscope according to claim 6, further comprising an immobile member including a space in which the second end portion of the first stem is housed, wherein lengths of the first stem and the second stem are set so that the second end portion and the first end face contact each other in the space of the immobile member.

10. An endoscope comprising:
a finger placement member having elasticity and disposed at an operation portion that is grasped by a user;
a first stem having a first longitudinal direction and having a first end portion connected inside the finger placement member;
a second stem having a first end face in contact with a second end portion of the first stem and slidably provided along a second longitudinal direction having a predetermined angle relative to the first longitudinal direction, the second end portion being opposite to the first end portion; and
a switch configured to be switched by the second stein sliding in the second longitudinal direction in contact with the second end portion of the first stem moving in conjunction with pushing of the finger placement member, wherein
a contact surface of the second end portion in contact with the first end face has a convex spherical surface shape; and
the first end face is a flat surface orthogonal to the second longitudinal direction.
